(19) United States
(12) Patent Application Publication
(10) Pub. No.: US 2004/0215139 A1
(43) Pub. Date: Oct. 28, 2004

(54) Apparatus and method for implanting left ventricular pacing leads within the coronary sinus

(76) Inventor: Todd Cohen, Port Washington, NY (US)

Correspondence Address: REED SMITH, LLP, ATTN: PATENT RECORDS DEPARTMENT, 599 LEXINGTON AVENUE, 29TH FLOOR, NEW YORK, NY, 10022-7650, US

(21) Appl. No.: 10/744,591

(22) Filed: Dec. 22, 2003

Related U.S. Application Data
(60) Provisional application No. 60/435,583, filed on Dec. 20, 2002.

(51) Int. Cl. A61M 31/00

(52) U.S. Cl. 604/95.04; 606/129; 607/4; 606/34

(57) ABSTRACT

A steerable catheter comprises a flexible tubular body having a proximal end, a distal end, and at least one lumen; an inflatable annular balloon positioned on or near the distal end of the tubular body; at least one electrode positioned on or near the distal end of the tubular body; and a handle attached to the proximal end of the tubular body. The handle cooperates with the distal end of the tubular body to steer the catheter. The catheter is especially useful in placing pacemaker or defibrillator leads in the heart or coronary sinus. Additionally, the invention also provides a device and platform for providing a variety of medical technologies including angiography, venography, angioplasty, stenting, valvuloplasty, embolization, drug delivery, and additional therapy delivery (e.g., laser, radiofrequency energy, ultrasound, microwave, etc).

### CROSS-REFERENCE TO RELATED APPLICATION

This application is based upon co-pending U.S. provisional patent application Ser. No. 60/435,583, filed Dec. 20, 2002, which is incorporated herein by reference in its entirety.

### FIELD OF THE INVENTION

The present invention relates generally to the design and use of medical devices. More particularly, the present invention relates to a steerable catheter which can be used for implanting cardiac pacemaker and defibrillation leads in the heart and coronary sinus. Additionally, the invention also provides a device and platform for providing a variety of medical technologies including angiography, venography, angioplasty, stenting, valvuloplasty, embolization, drug delivery, and additional therapy delivery (i.e., laser, radiofrequency energy, ultrasound, microwave, etc).

### BACKGROUND OF THE INVENTION

Implantable pacemakers and defibrillators have adapted a therapy of pacing both the right and left ventricles simultaneously or closely separated (i.e., sequentially) to optimize cardiac performance in patients with severe congestive heart failure. This technique, called cardiac resynchronization therapy or biventricular pacing, is often performed by percutaneously placing pacing leads in the right heart (i.e., a right ventricular lead and possibly a right atrial lead to accomplish atrio-ventricular (AV) sequential pacing). In addition, a pacemaker lead is also placed into the left ventricle via a percutaneous procedure in which the operator places an additional pacemaker lead into a left ventricular branch of the coronary sinus vein the blood vessel which leads from the bottom of the right atrium near the right ventricle and travels to the left side of the heart.

Implantable cardiac pacemakers and/or defibrillators require placement of electrical lead wires within the heart or coronary sinus, where the electrical leads may be connected to a remotely implanted pacemaking unit. Placement of the electrical lead wires can be accomplished either by open surgical techniques or by transvenous techniques. Transvenous lead placement is accomplished by establishing percutaneous access to the venous system, typically via the subclavian vein, and passing the electrical lead to the desired target location within the heart, usually the right ventricular apex. The lead will include a self-anchoring mechanism at its distal end, such as a helix, screw, or tines, and the end of the lead wire can be engaged against and anchored in the endocardium.

Recently a trial entitled COMPANION demonstrated that cardiac resynchronization therapy can reduce mortality plus hospitalization by 20% in patients with New York Heart Association class III and class IV heart failure, a left ventricular ejection fraction of <36% and a QRS duration of 120 ms. The latter two findings are frequently observed in such heart failure patients.

The most difficult part of the procedure of implanting a biventricular pacemaker and/or defibrillator is the implantation and deployment of the left ventricular or coronary sinus lead, which is especially difficult and may be very time consuming in patients with very sick and severely dilated hearts. There are at least five obstacles which must be overcome to successfully place these leads:

Percutaneous access through a chest wall vein (subclavian or cephalic vein).

Finding the coronary sinus vein and placing an introducer sheath in the vessel.

Performing a venogram by placing a balloon catheter down the introducer sheath inside the proximal end of the coronary sinus. The distal end of the balloon catheter will be advanced distally to the distal end of the introducer sheath inside the coronary sinus. The balloon is then inflated to occlude the blood vessel, and contrast dye is administered through the catheter's lumen to image the blood vessel and find the precise location of its branches.

Withdrawal of the balloon catheter and placement of the left ventricular pacing lead through the introducer sheath possibly using a guidewire (over-the-wire system) and advancing the lead into the optimal left ventricular branch.

Withdrawal of the introducer sheath over the pacing wire, after which the leads are secured and attached to the device.

Alternatively, the leads can be positioned using an internal stiffening stylet which is used to guide the distal end of the lead under fluoroscopic imaging. Since the leads lack column strength and there is substantial blood flow through the heart chambers, manipulating a lead is difficult and positioning of the lead is not always accurate. Thus, the physician must often disengage the lead anchor and reposition the lead one or more times before satisfactory placement is completed. It will be appreciated that such repositioning of the electrical lead can cause unnecessary trauma to the patient, with risk of causing arrhythmias and ventricular perforation.

An additional problem with pacemaker and/or defibrillator leads as they exist is that their current design consisting of a central lumen for stylet placement for postioning, may make these leads more prone to damage. Newer leads, with smaller internal diameters, and perhaps no central lumen may be more durable. The delivery of said leads would require another modality other than the traditional stylet.

Similarly, there are other areas in cardiology, radiology, and internal medicine etc. where many which utilize venography, angiography, angioplasty, stenting, valvuloplasty as well as other therapy delivery where it is difficult to move from point A to point B through circuitous corporeal or circulatory systems.

There is a tremendous need for an easy device and method to help doctors implant leads for pacemakers and defribillators throughout the heart. In addition, the same difficulty that exists with pacing and defibrillators also exists in other areas of electrophysiology, cardiology, and interventional radiology as well as other areas of medicine. In summary, there is an overall need to improve upon the ease of localization, visualization, and delivery of a plurality of therapies throughout medicine.

### OBJECTS OF THE INVENTION

It is an object of the invention to provide a catheter for implanting cardiac pacemaker and defibrillator leads.

It is also object of the invention to provide a catheter and platform for implanting cardiac pacemaker and defibrillator leads in branches of the coronary sinus vein.

It is a further object of the invention to provide a sheath for implanting cardiac pacemaker and defibrillator leads in a variety of locations throughout the myocardium.

It is also an object of the invention to provide a catheter to map and perform pulmonary vein angiography.

It is a further object of the invention to provide a catheter and method to perform a variety of venograms and angiograms throughout the circulatory system, including but not limited to, the myocardium and coronary arteries, renal arteries, aorta, and the pulmonary artery.

It is a further object of the invention to provide a catheter and method to perform angiography.

It is a yet further object of the invention to provide a catheter and method to perform an embolization.

It is a yet further object of the invention to provide a catheter and method to perform an angioplasty.

It is a yet further object of the invention to provide a catheter and method to place a stent in a patient.

It is a yet further object of the invention to provide a catheter and method to treat a lesion in a patient's gastrointestinal tract.

It is a yet further object of the invention to provide a catheter and method for urological or nephrological applications.

It is a yet further objection of the invention to provide a steerable catheter with distal electrodes and a working lumen for different cardiovascular or other corporal functions.

It is a yet further object of the invention to provide a catheter and method to treat a lesion, perform an endoscopy and/or biopsy and/or deliver a stent in a patient's gastrointestinal tract and/or a mechanism for performing endoscopic resections (ERCP).

It is a yet further object of the invention to provide a catheter and method for urological or nephrological applications including cystography, dilation, biopsy, and stent delivery and performing therapy delivery.

It is a yet further objection of the invention to provide a steerable catheter with or without distal electrodes and a working lumen for different cardiovascular or other corporeal functions.

These and other objects of the invention will become more apparent from the discussion below.

### SUMMARY OF THE INVENTION

According to the present invention, a catheter is provided for the transvenous placement of intracardiac electrical lead wires useful for connection to implantable pacemakers, defibrillators, and other electrophysiology devices. The catheter is steerable, to permit accurate positioning of its distal end at a target site within a heart chamber or vessel. Most commonly, the target site will be the right ventricular apex, but the target site can also be the coronary sinus, tricuspid annulus, atrial appendage, atrial free wall (in patients with no atrial appendage), or the like. A balloon is provided at the distal end of the catheter to permit atraumatic engagement of the catheter against the endocardium at the target site and also to help anchor and/or secure the location of said catheter. In addition, the balloon will help occlude a vessel such as the coronary sinus vein in order to help visualize a target vessel via contrast venography. The catheter may be placed into the circulatory system directly over a guide catheter or through an introducer sheath. After the distal end of the catheter has been properly placed and placement confirmed, a conventional electrical lead wire may be introduced through the lumen of the catheter or through the introducer sheath which is advanced over the catheter. This steerable electrophysiology catheter can help define the location of the coronary sinus vein electrically and via contrast injection through the lumen.

A catheter according to the invention comprises at least one lumen and an inflatable balloon at the distal end of the catheter. At least one electrode, preferably two, is positioned on the outer surface of the catheter distal to the balloon, and each electrode is connected to a proximally extending wire. Preferably the wires extend longitudinally through a lumen or the sidewall of the catheter.

A central lumen extends the length of the catheter, and a steering or pull wire(s) or cord(s) extends from a position at or adjacent to the distal end of the catheter proximally to a steerable hub or handle. The steering or pull wire(s) or cord(s) preferably extends through a lumen, optionally the same lumen through which each wire for an electrode extends. The distal handle, hub, or knob may consist of additional movable knobs, levers, etc for providing catheter tip deflection from straight to fully curved. Another embodiment may consist of some or all of the following additional controls for tightening and loosening said deflection, a control for changing the point of deflection and thereby altering the radius of curvature, the option to create secondary bends and shapes on said catheter, as well as a rotational mechanism.

The catheters of the invention may or may not have a pre-set distal curve, and different catheters may have curves with different radii of diameter, referred to as “small”, “medium”, “large”, or “extra large.” The catheter curve characteristics can be due to pre-forming of the catheter or the way in which the steering or pull wire or cord is arranged, or both. Optionally the catheter is steerable with inherent means to control the radius of curvature of the distal portion. In addition, other deflections and curvatures can be achieved by additional pull wires, levers, and mechanisms concealed within said catheter.

The catheter can come in different lengths, which can be utilized for different purposes. For example, a long version can be utilized to perform coronary sinus venography from the groin (i.e., femoral vein) to visualize the vessel prior to surgery. This could be performed during an electrophysiological procedure/study. The catheter could be positioned in the conventional way from the groin in which the catheter is advanced from the femoral vein through the peripheral circulation into the right side of the heart. In the right atrium the tip of the catheter is curved and pointed toward the lower septal region to engage the coronary sinus ostium. The catheter is then rotated such that the catheter advances freely into the coronary sinus. The catheter is positioned in the proximal component, and a solution, for example, 10 to 20 cc of contrast is administered to visualize the vessel and its branches.

A shorter version is available to simplify coronary sinus access for lead placement. By use of a long straight sheath a large curved catheter is placed and positioned in the coronary sinus. The introducer is then advanced over the catheter into the very proximal region of the vessel. The catheter then can be advanced into the vessel beyond the sheath and the balloon inflated to perform venography. The electrodes can help with visualization of the catheter both flouroscopically and electrically. The catheter can then be removed and the left ventricular pacing lead could then be placed and positioned accordingly in a lateral left ventricular branch. This could be placed directly or via an exchange mechanism/wire.

The sheath is then removed over the lead and the connector of the lead is eventually attached to the implantable device.

The longer version of the catheter can be used from the groin without the electrodes. The same catheter with specially designed inflatable angioplasty balloons (noncompliant balloons) and/or stent-deploying system can be used for peripheral interventions (renal artery stenosis, etc). In addition, a larger sized (noncompliant) balloon could be adapted and used for mitral and aortic valvuloplasties.

The longer version of the catheter can also be used to help map, visualize, electrically isolate and ablate left-sided pulmonary veins. This is accomplished by positioning the catheter across the interatrial septum (transeptal procedure). The catheter can steer into all four plus pulmonary veins, and venograms could be performed. Electrodes can also be arranged both proximally and to help determine complete electrical isolation of the pulmonary vein potentials via bidirectional block techniques.

The steering mechanism will prove useful for engaging and mapping the more difficult pulmonary vein locations.

### BRIEF DESCRIPTION OF THE DRAWINGS

FIG. 1. is a partially cross-sectional view of a catheter embodiment of the invention.

FIG. 2. is a cross-sectional view across line 2-2 of the embodiment of the invention in FIG. 1.

FIG. 3. is a partial sectional view of the proximal end of the proximal hub or handle of the embodiment of the invention in FIG. 1.

FIG. 4 is a perspective view of another embodiment of the invention.

FIG. 5 is a perspective view of a variation of the distal portion of the embodiment of the invention shown in FIG. 4.

FIGS. 6 to 9 illustrate the placement of a left ventricular (LV) lead in the coronary sinus vein.

FIG. 10 is a partial cross-sectional view of an embodiment of the invention that comprises an introducer sheath.

FIG. 11 is a cross-sectional view across line 11-11 of the embodiment of the invention shown in FIG. 10.

### DETAILED DESCRIPTION OF THE INVENTION

A steerable lumen catheter and method designed to engage and access the coronary sinus vein is described. Such a catheter has a balloon towards its distal tip which, if inflated, can secure the catheter within the proximal vein and permit occlusive venography/angiography of the vessel. The lumen can also permit the delivery of a soft floppy guidewire into a specific branch of the coronary sinus vein for exchange with an over-the-wire pacing catheter. Distal electrodes on the catheter help define the atrioventricular groove and permit pacing and sensing as necessary. The catheter as designed can be used to permit pulmonary venography and recording of pulmonary vein potentials. In addition, this catheter can be used in interventional radiology to steer the lumen catheter into segmental vessels and provide selective angiography (perhaps in a catheter as described without the electrodes, or the electrodes can serve as opaque fluoroscopic markers or tissue contact evaluators). With an attached angioplasty and stenting high pressure balloon the catheter can also provide therapy, for example, to treat stenosis in an occluded vessel such as a renal artery. Also, the catheter could be designed with a large enough central lumen to permit direct placement of the pacemaker lead in the coronary sinus. In addition, a version of the above catheter which is actually the coronary sinus pacing lead with a removable handle (with or without the balloon) is also feasible, to streamline said procedure. The catheter is also useful in gastrointestinal and nephrological applications, as discussed below in more detail.

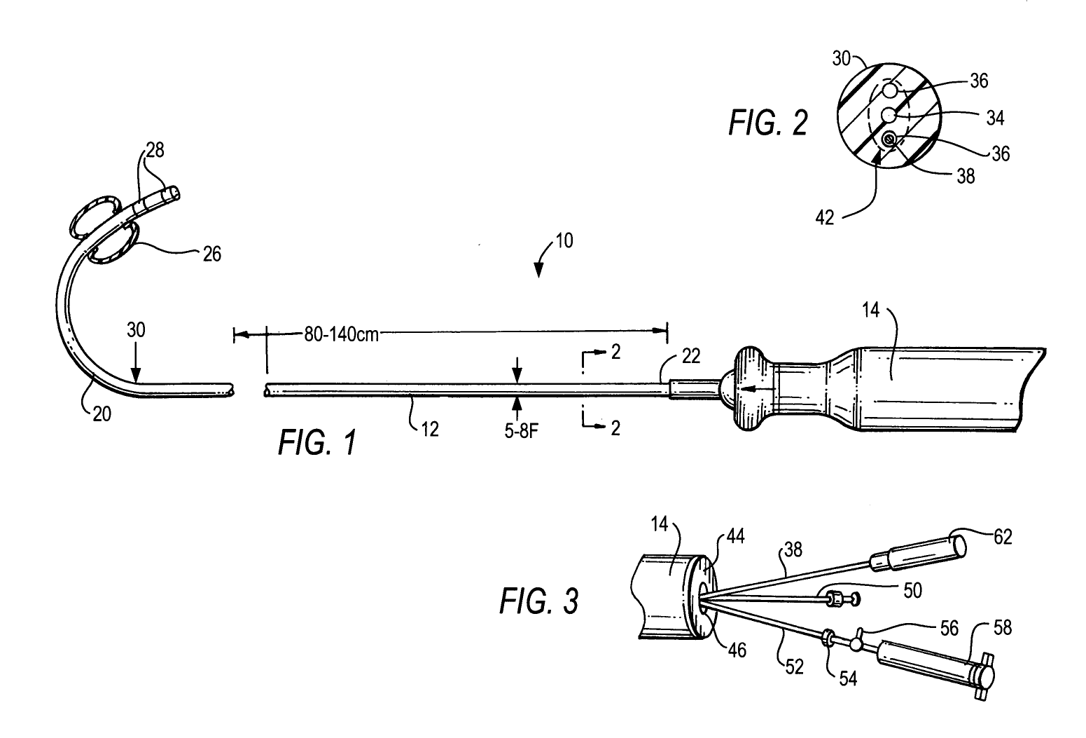

The invention can perhaps be better understood by referring to the drawings. As shown in FIG. 1, a catheter 10 has a longitudinally extending member 12 and a hub or handle 14. Longitudinally extending member 12 has a distal section 18, an intermediate section 20, and a proximal section 22. Distal section 18 has an annular inflatable balloon 26 and one or more, preferably two, electrodes 28.

Distal section 18 and intermediate section 20 may optionally have a curvature which varies from catheter to catheter or is varied by the operator. From a point 30 between intermediate portion 20 and proximal portion 22, the curvature of intermediate portion 20 will be small, medium, large, or extra large, dependent upon the effective radius of curvature. Additional curvatures such as those that are dependent on the superior vena cava, or more traditional multipurpose and/or Amplatz curvatures are feasible. The curvature can either be pre-set or pre-formed, or it can result from mechanical or other constraints due to wires or other “hardware” within the catheter, or both. Since preferably the catheter is a steerable catheter that has an internal steering or pull wire(s) or cord(s), the curvature of distal 16 and intermediate section 20 could be connected to or in functional communication through the steering or pull wire(s) or cord(s) with hub or handle 14.

In the cross-section of FIG. 2, proximal section 30 has three lumens, a central lumen 34 and two ancillary lumens 36. One of ancillary lumens 36 is an inflation lumen for, and in fluid communication with, balloon 28, and the other lumen 36 is for the wires 38 extending from the electrodes 28 and a steering or pull wire or cord 42 that extends to distal section 18.

Proximal catheter section 22 is attached to hub or handle 14, which is preferably ergonomic. As shown in FIG. 3, the proximal end 44 of hub or handle 14 has an opening 46 through which extend the proximal ends of wires 38, an access 50 to central lumen 34, and an access 52 in fluid communication with an inflation lumen 36. Access 52 has a side port or Luer Lock or other connector 54, optionally with a stopcock 56, for connection to an inflator 58. The distal ends of wires 38 end in an electrical connector 62.

Preferably hub or handle 14 is connected to steering or pull wire(s) or cord(s) 42, and is configured so that rotational or other movement of hub or handle 14 causes longitudinal member 12 to rotate and/or distal portion 18 and intermediate portion 20 to bend or straighten. More particularly, hub or handle 14 has a distal section 64 that may function to rotate longitudinally extending member 12 and/or engage steering or pull wire(s) or cord(s) 42 to “steer” distal section 18.

Steerable catheters are well known to those skilled in the art, and the particular steering and/or bending system employed to steer, that is, rotate and/or bend, the distal section of the catheter is not critical. See for example, U.S. Pat. Nos. 4,723,936, 4,757,827, 5,372,587, 5,449,343, 5,562,619, 5,935,102, 6,120,516, 6,241,667, 6,485,455, 6,491,681, and 6,530,913, all of which are incorporated herein in their entirety by reference. What is important is that the distal portion 18 of the catheter and the hub or handle 14 cooperate and/or are connected and/or communicate to steer the catheter effectively to accomplish the intent of the invention. Effective steering would comprise rotation of the intermediate and/or distal sections of the catheter as well as curvature, preferably adjustable curvatures, of the catheter distal section.

The length and diameter of catheter 10 could vary dependent upon the particular application, but it is believed that one skilled in the art could determine appropriate parameters. For example, catheter proximal section 22 is likely to be from about 20 to about 140 cm in length, with a diameter of from about 2 to about 12 F, preferably from about 5 to about 8 F. Intermediate section 20 is probably from about 10 to about 50 cm in length, and distal portion 18 is preferably from about 10 to about 40 cm in length, with a total length of from about 40 to about 200 cm. The electrodes 28 are each about 1 to about 4 mm, preferably from about 1 to about 2 mm, in width, and the lumens 34, 36 within catheter 10 are each about 0.01 to about 0.4 inches, preferably from about 0.01 to about 0.25 inches, in diameter. This steerable electrophysiology catheter would come in two sizes: Regular: 40-110 cm for the chest/upper extremity vein access procedures and a longer length (90-200 cm) for groin access through the femoral vein or artery.

More particularly, this patent application describes an easily steerable electrophysiology catheter with a lumen and inflatable balloon as well as a technique, which vastly simplifies the finding of the coronary sinus vein. In addition, this catheter can permit an occlusive venogram of a blood vessel without removal of the catheter.

A novel method, according to the mention that uses this steerable lumen catheter with a balloon, in an electrophysiology application comprises the following steps:

1; Obtain access of the blood vessel and place a long straight removable sheath into the right atrium.

2. Place the steerable catheter into the long sheath. Position the catheter out of the sheath and pull a curve on the catheter. Move the catheter in and out until the coronary sinus vein is cannulated. The left anterior oblique location on fluoroscopy is often best to help identify the location. Confirmation in the right anterior oblique position is also useful. Then, advance the long sheath over the catheter (much like the Seldinger technique) such that the distal sheath is approximately 1 inch within the coronary sinus vein. The catheter then could be pulled back such that the balloon is outside the sheath inside the coronary sinus vein and the balloon could then be inflated to occlude the blood vessel. Contrast dye could then be administered through the central lumen of the steerable catheter and a venogram obtained. This should only be performed if the patient has substantially normal renal function or is on dialysis. After the venogram identifies a target branch of the coronary sinus, the catheter is then removed from the sheath.

3. The left ventricular pacing lead is then placed into the sheath and advanced (possibly using a guidewire) into the appropriate left ventricular branch.

4. The sheath is then removed, and the wires are secured and attached to the device.

In one embodiment of the invention, a method for implanting a pacemaker lead inside the coronary sinus vein to pace the left ventricle of the heart, comprises the steps of:

percutaneously inserting an long introducer sheath into the subclavian vein (cepahlic vein or equivalent);

advancing a steerable lumen catheter through the introducer into the right heart;

placing a curve on the catheter once outside of the distal end of the introducer sheath;

moving the catheter backwards and forwards to cannulate the coronary sinus vein under fluoroscopy;

varying the curvature of said catheter while moving it back and forth to find the coronary sinus vein ostium;

optionally injecting puffs of contrast through the lumen of said catheter to help confirm that one is inside the coronary sinus vein;

exchanging said catheter for a different curvature to cannulate the coronary sinus if necessary (i.e., small, medium, large, and extra large curvatures);

advancing a long sheath over the catheter once well inside the coronary sinus vein to have stable access to the coronary sinus;

pulling out the catheter and placing a pacemaker lead into the introducer sheath and directing it into a branch of the coronary sinus vein which is a left ventricular branch;

checking pacing and sensing thresholds of said pacemaker lead and establishing adequate left ventricular pacing;

removing said sheath over the lead while maintaining the lead in stable position within the coronary sinus vein branch; and

securing said catheter to the floor of the pacemaker pocket and attaching the lead to a device to help pace the heart.

In another embodiment of the invention, a method for implanting a pacemaker lead inside the coronary sinus vein comprises the steps of:

use of a steerable lumen balloon tip catheter;

cannulation and placement of a catheter of the invention in claim 1 and advancement of said sheath beyond the coronary sinus ostium to secure the sheath;

pulling back said catheter such that the balloon tip is just outside the introducer sheath which lies in the proximal component of the coronary sinus vein;

inflation of the balloon to occlude the proximal portion of the coronary sinus vein;

injection of contrast through the lumen of said catheter to fluoroscopically image the coronary sinus vein and its branches;

removal of said catheter from the introducer sheath;

placement of a pacemaker lead into said sheath and advancement into a left ventricular branch of the coronary sinus;

testing the pacing and sensing function of said lead to assure adequate thresholds and function;

removal of said sheath over the lead while maintaining the lead tip in the appropriate location;

securing the lead end to the floor of the devise pocket; and

attaching lead to device.

In yet another embodiment of the invention, a method for implanting a pacemaker lead inside the coronary sinus vein to pace the left ventricle of the heart, comprises the steps of:

percutaneously accessing an upper chest, extremity or neck vein;

placing an introducer into said vein;

inserting a steerable pacemaker lead catheter system which is comprised of:

2-6 F pacemaker lead with an axial lumen and bipolar electrodes at the tip;

a proximal connector with an internal lumen with locking or screw-in stylet which is used as a pull cord for steering said lead; and

a removable handle with a steerable mechanism for pulling and releasing said stylet to steer said lead into the branch of the coronary sinus vein;

advancing said steerable lead system with attached handle into said introducer and moving it back and forth with varying curvatures until the coronary sinus ostium is engaged;

optionally administering puffs of contrast to visualize location;

optionally using the electrodes to help define location once connected to an electrogram recording system;

once in the coronary sinus removing the stylet and handle and placing a soft steerable guidewire through the central lumen and advancing it into an appropriate coronary sinus branch;

advancing the pacing lead and then following said guidewire to the intended location to pace the left ventricle;

removing the guidewire;

testing the pacing and sensing to confirm appropriateness;

if in the step above the pacing and sensing are not appropriate, the lead is pulled back and the guidewire is positioned in another location and the procedure repeated until adequate pacing and sensing are achieved;

removing the sheath over the lead while making sure the tip of the lead does not dislodge;

securing the proximal area of the lead to the floor of the device pocket using a lead sleeve or bullet to protect the lead; and

attaching the lead to the device and the remainder of the leads, etc.

Preferably the left ventricular pacing lead uses conventional nonlocking or screw-in stylets optionally curved manually to direct the lead into the coronary sinus vein.

It should be noted that 3 to 4 different curvatures of the steerable lumen catheter can be used in electrophysiologic applications, which includes small, medium, large, and/or extra large curvatures. Preferably one should start with a straight sheath and a large curvature. If the coronary sinus vein cannot be engaged with this system within 5 to 10 minutes, another curvature should be considered (e.g., extra large curve). In addition, puffs of contrast can be given from the tip of the catheter to see if it is in the coronary sinus ostium. Additionally, steerable sheaths could also be employed to help access the coronary sinus vein.

In addition to the above device and method a slight modification on the device could permit:

1. Pulmonary Venography with recording of potentials within the pulmonary vein. Such a catheter could also help elucidate complete pulmonary vein isolation or block during catheter ablation procedures.

2. A steerable catheter which could be used to steer the catheter across peripheral occlusions such as renal artery stenosis. With a high pressure balloon angioplasty and stent deployment could be facilitated.

3. Lastly, a similar design could help facilitate mitral and aortic valvuloplasty by steering the catheter near the stenotic valve. After passing a guidewire, and then inflating the high pressure balloon, the stenostic valve can be opened accordingly.

In addition, the above catheter could be designed such that the central lumen is large enough (2 to 6 F) to directly deliver the left ventricular pacing wire.

Finally, a streamlined version of this invention replaces the steerable catheter as described above with the coronary sinus pacemaker lead. In this embodiment of the invention the pacing lead consists of an internal stylet which could be connected to a removable distal handle to steer the catheter directly into the coronary sinus. The pacing lead has an axial lumen for 1) the stylet, 2) the guidewire, and 3) ability to deliver puffs of contrast for venography/identification that the catheter is in the coronary sinus. In essence the pacing lead has many of the same components of the steerable lumen EP catheter including the distal bipole for pacing and sensing.

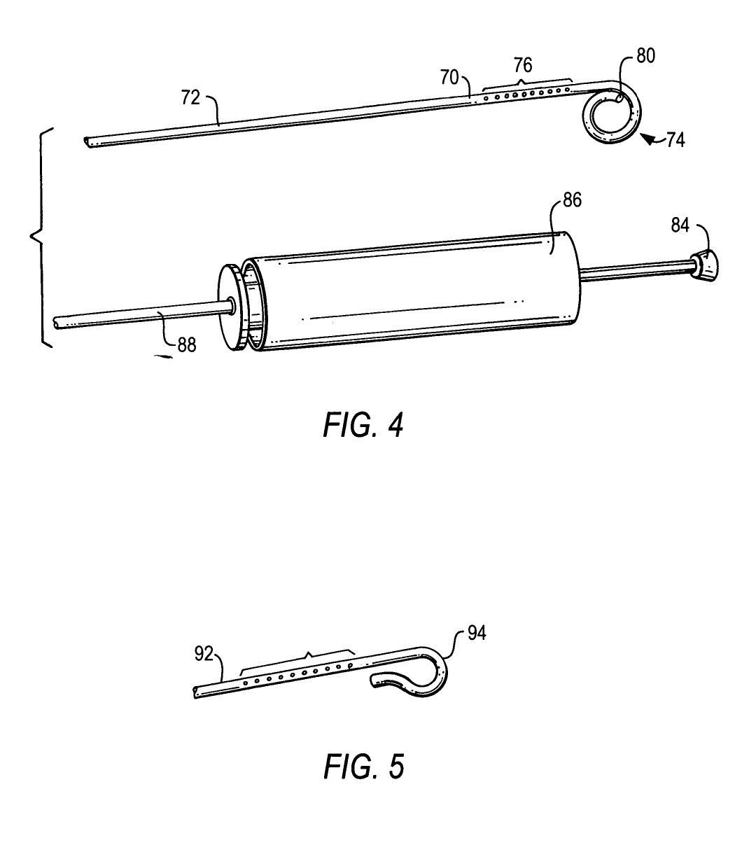

In another embodiment of a catheter of the invention as shown in FIG. 4, the distal section 70 of a catheter 72 may comprise at least one steering or pull wire or cord (not shown) for steering the catheter and a “pigtail” configuration 74. Distal section 70 comprises a plurality, perhaps from 4 to 12, preferably 8 or 10, lateral openings or side holes 76 for delivering fluid or solution therapy. An axially extending lumen extends from distal tip 80 of catheter 72 to and through an access 84 proximal to handle 86 at the proximal end 88 of catheter 72. Handle 86 engages each steering or pull wire or cord to cause the distal section 70 to bend or deflect.

In a variation of this embodiment of the invention as shown in FIG. 5, catheter distal section 92 may have a slightly more open configuration 94. It is constructed so that if a guidewire (not shown) were inserted through the axial lumen of the catheter, configuration 94 would straighten to enable the operator to select a vessel branch. It is within the scope of the invention that the catheter distal tip have have a slight bend of greater than at least about 0°, up to at least about 180°, to at least about 270°, to the extent that the distal catheter configuration will be a spiral or pigtail.

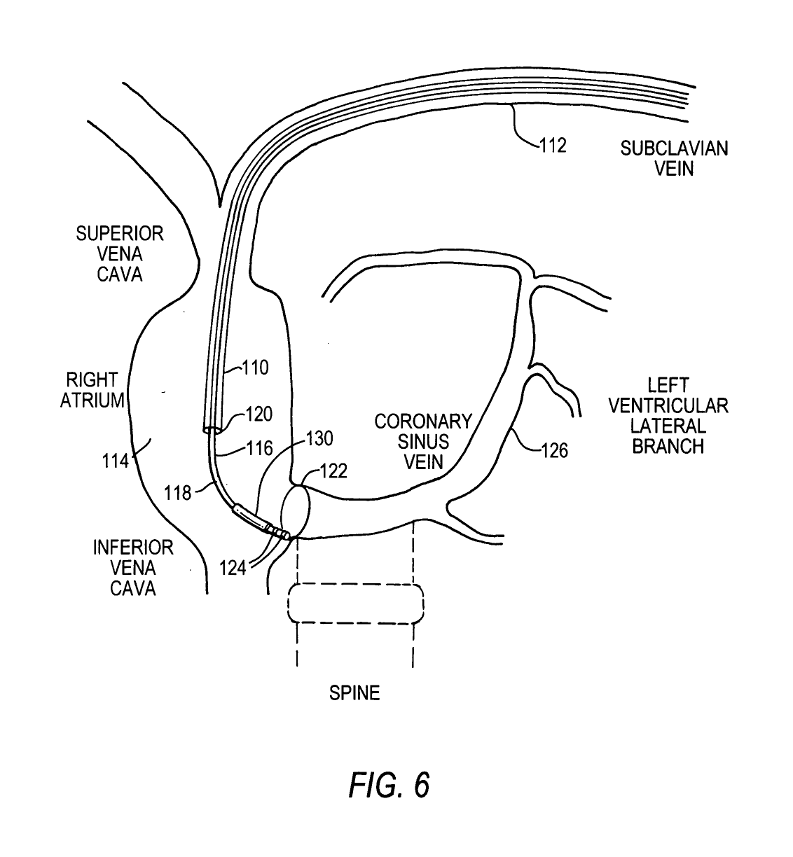

With reference to FIGS. 6 to 9, use of a catheter for introducing an electrical lead is shown. Initially an introducer sheath 110 is percutaneously introduced through the subclavian vein 112 to the right atrium 114. Then, a catheter 116 according to the invention is advanced through the introducer sheath 110 so that the distal portion 118 of catheter 116 is distal to the distal portion 120 of introducer sheath 110. To identify the coronary sinus ostium 122 of the coronary sinus vein 126, the operator should use electrodes 124 on catheter distal portion 118, contrast, and/or fluoroscopic anatomical information to identify the coronary sinus ostium 122. Catheter 116 preferably has an inflatable balloon 130.

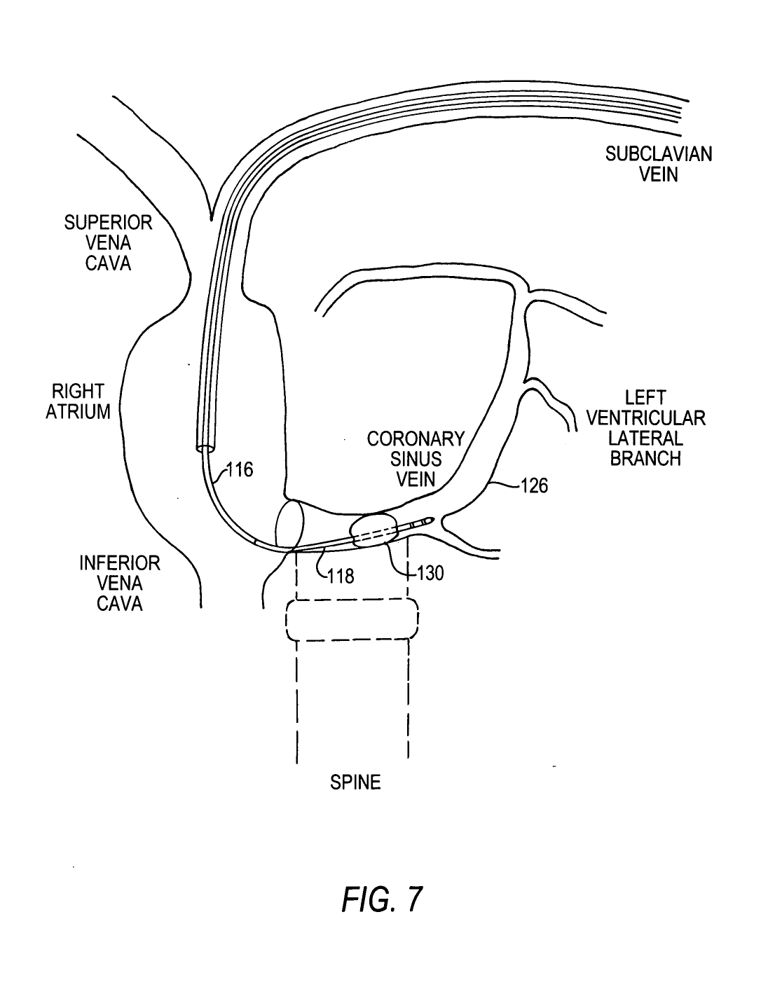

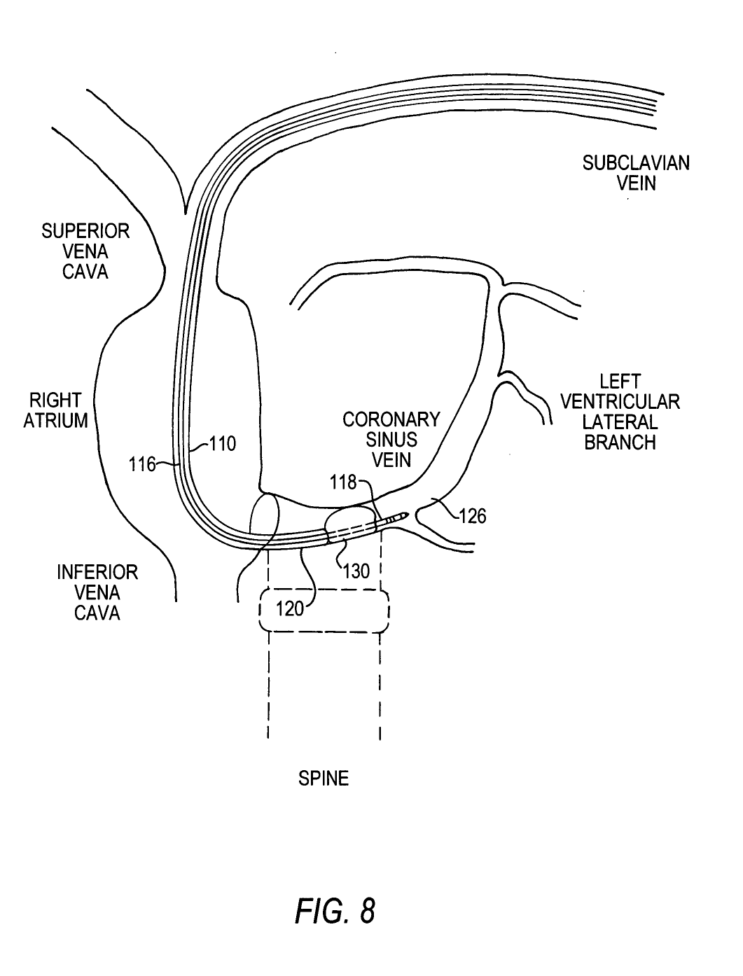

In FIG. 7 the distal portion 118 of catheter 116 has been advanced into the coronary sinus vein 126. Optionally balloon 130 can be inflated for an occlusive venogram. In a next step, as shown in FIG. 8, introducer sheath 110 is advanced over catheter 116 so that the distal portion 120 of introducer sheath 110 is adjacent to balloon 130. Preferably the distal portion 120 of introducer sheath 110 will be well seated to deliver a guidewire or LV lead. Balloon 130 is deflated and catheter 116 is withdrawn proximally.

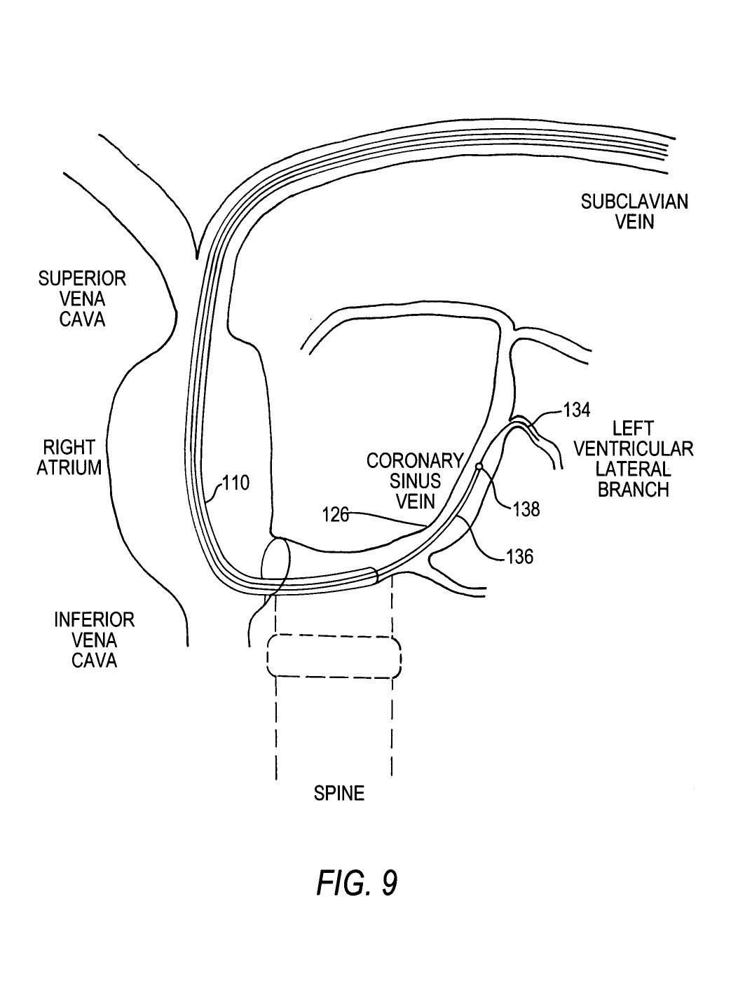

As shown in FIG. 9, a guidewire 134 can be advanced through introducer sheath 110 into the coronary sinus vein 126 and then an LV lead 136 can be advanced over guidewire 134 or, dependent upon the particular lead, LV lead 136 could itself be advanced through introducer sheath 110 without a guidewire. Use of a guidewire would be preferred, especially for guiding the LV lead to a left ventricular arterial branch. Preferably LV lead 136 has an anchor 138 at its distal end to be engaged against and into the endocardium or the vessel wall. Alternatively, a predetermined lead curvature or larger tip could help stabilize said lead within said vessel. Anchor 138 may be in the form of a helix, a tine structure, or any other conventional self-anchoring mechanism.

Once the distal portion of the left ventricular lead is properly positioned, introducer sheath 110 and/or guidewire 134 can be withdrawn.

It is within the scope of the invention that the steerable catheter itself can be used as a left ventricular lead. The distal portion of the catheter would be advanced into the coronary sinus vein to the extent that the electrodes on the distal portion of the catheter would be at substantially the same position as the distal portion of a left ventricular lead would be. The catheter so used would be with or without an annular inflatable balloon.

As mentioned above, a steerable catheter according to the invention can be useful for other procedures. In one embodiment, the steerable catheter would be used to perform venography, angiography, embolization, angioplasty, and/or stenting, procedures well known to those skilled in the art. For example, the steerable catheter would be advanced through an introducer sheath into a patient's left coronary artery and then contrast would be injected for an angiography. If a blockage is identified, a guidewire is advanced through the central lumen of the catheter and through the vessel with the blockage, or stenosis, so that the guidewire distal portion is across and distal to the stenosis, and the catheter is advanced over the guidewire to position the inflatable balloon within the stenosis. The balloon is inflated to open the vessel. Optionally, the first catheter is withdrawn and then another catheter having an expandable stent is advanced over the guidewire to position the balloon/stent at the stenosis site. The second balloon is inflated to position the stent, the balloon is deflated, and then the catheter and guidewire are withdrawn. The above procedure could be accomplished with a single catheter which could not only pass through a vessel's obstruction but also deploy the stent.

The steerablility of said system via the handle would be particularly useful in tortuous vasculature and coronary anatomy. The handle, levers, and steering mechanisms would make an otherwise multicatheter/guidewire procedure quicker and easier. One catheter would be able to change its shape, curvature and configuration in order to negotiate and find the target vessels and or lesions.

In another embodiment of the invention a steerable catheter is used to map and perform a pulmonary venogram. The distal portion of the catheter is steered trans-septally from the right atrium to the left atrium and then into the pulmonary vein. The balloon is inflated and then contrast is injected. Also, with or without the balloon inflated pacing from a proximal to a distal electrode or pairs of electrodes can be used to determine lines or areas of conduction block.

In a further embodiment of the invention a pulmonary angiography is performed in the pulmonary artery by positioning a steerable catheter according to the invention from the femoral vein into the right heart across the pulmonary valve into the pulmonary artery. Selective angiography can be performed by identifying a branch, inflating the distal inflatable balloon, and injecting contrast. Any obstructions or blockages determined can be relieved by selective thrombosis, e.g., drug delivery through the catheter lumen, angioplasty, and/or stent delivery with a noncompliant balloon.

In a further embodiment of the invention, a steerable catheter according to the invention can be used to open a blockage in a renal artery. Similar to the procedure described above, the catheter is advanced to the blockage, the balloon is inflated, and then a second catheter delivers a stent.

In a further embodiment of the invention a steerable catheter of the invention is used to perform embolization of collagen or thrombus or similar substances can be deployed to infarct a variety of tissues or organs.

In a further embodiment of the invention a steerable catheter of the invention is used to treat benign prostatic hypertrophy (BPH). The catheter is advanced into a patient's urethra to position the inflatable balloon within an enlarged prostate, whereby the balloon is inflated to relieve narrowing of the urethra. If an expandable stent was carried on the balloon of that catheter, the stent will remain in an expanded state. Alternatively, the first catheter may be withdrawn and a second catheter advanced with a stent. A single catheter could accomplish both functions. An obstruction in a patient's urethra can be treated in similar fashion. Also, in either situation ultrasound or other energys can be delivered as well.

In a further embodiment of the invention a steerable catheter of the invention is used to treat esophageal strictures. The catheter is directed, optionally through an introducer sheath, to an area of stricture, where the balloon is inflated to treat a lesion. Optionally a stent can be employed or ultrasound or other energy can be delivered.

In a further embodiment of the invention a steerable catheter of the invention can be used to deploy a biliary stent to treat cholelithiasis.

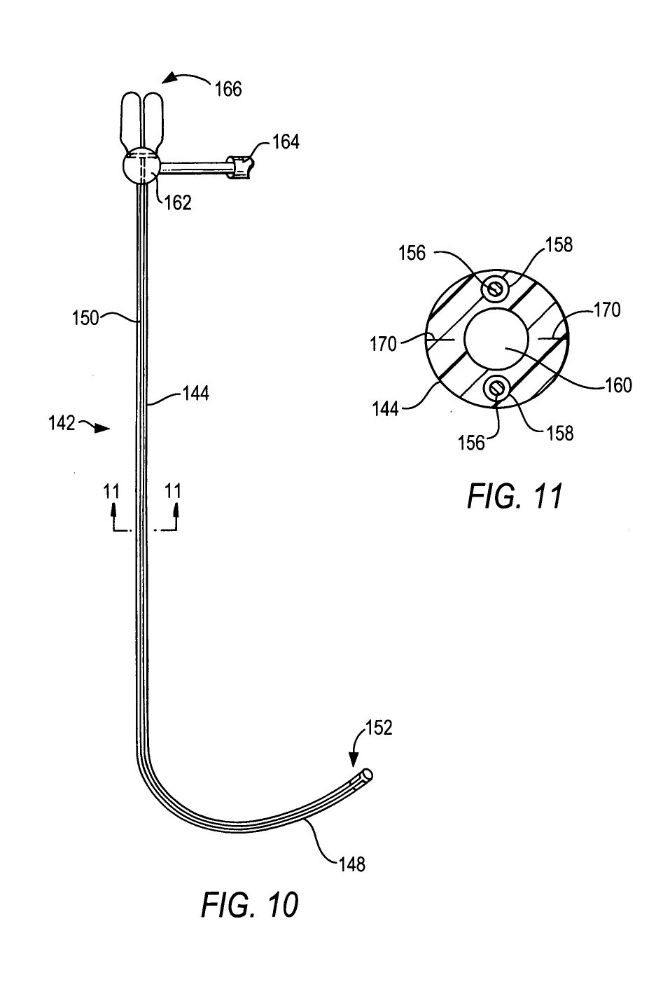

In a further embodiment of the invention, as represented in FIGS. 10 and 11, an introducer sheath comprises a structure similar to that described in FIGS. 1 to 3. The introducer sheath 142 comprises a longitudinally extending member 144 having a distal section 148 and a proximal section 150. The distal section 148 comprises one or more, preferably two, electrodes 152.

Two electrodes 152 can create a bipole for recording an atrioventricular electrogram. Also, when two electrodes 152 are used, they are preferably oppositely positioned and each connected to a proximately extending wire 156 that extends through each lumen 158 adjacent axial lumen 160, as shown in FIG. 11.

The proximal section 150 comprises a hub 162 that preferably has a hemostatic valve or diaphragm to prevent blood backbleeding. Also, hub 162 has at least one port 164, for example, a port connected to a stopcock (not shown) to introduce flushing solution.

Hub 162 and longitudinally extending member 144 terminate in splitter tabs 166, preferably plastic. Tabs 166 are intended to divide/split in the middle, and, as tabs 166 are pulled essentially in a direction perpendicular to the longitudinal axis of longitudinally extending member 144, introducer sheath 142 splits apart longitudinally along axial lines of weakness 170.

Each electrode 152 has a proximally extending wire 162. In a preferred embodiment having two electrodes 152, each electrode 152 will have a wire 162 extending through an oppositely positioned lumen 166. Then, when introducer sheath 142 is split, each half will have an intact electrode/wire system.

Introducer sheath 142 can have different shapes or shaping means. The introducer can be pre-formed to be straight, to be Amplatz-shaped, or to have an extended hook shape. Alternatively, the introducer can be steerable/adjustable where the distal section can be adjusted to change its radius of curvature, as described above for the catheter. To accomplish this hub 156 will also comprise means to engage and/or activate and/or communicate with a steering or bending wire or cord. Optionally introducer sheath 142 may have an inflatable balloon, which would be in fluid communication with an inflation lumen.

It is within the scope of the invention that the inflatable balloon can also deliver a fixative, such as ethanol, into heart or vessel tissue to create a discrete line of electrically inactive tissue.

It is within the scope and mechanism to prevent the back flow of blood and stabilize the pacing lead such that it does not move while inserting the sheath into the vascular system. By untightening the valve mechanism the lead can be advanced into the heart and coronary sinus vein. The wires and electrodes of the preloaded steerable sheath contained in the pacing lead permit easy visibility of said sheath. The pacing lead can be advanced into the coronary sinus vein, and via connector electrodes from the distal end atrioventricular electrograms consistent with an atrioventricular location can be identified thereby confirming the coronary sinus location without a contrast injection. In another embodiment, the pacing lead can also be preloaded with a 0.018 or 0.014 inch guidewire with the wire also at the distal tip of said system ready to advance this sheath directly into the coronary sinus vein. Using this method one can achieve venous access by standard methods, place the sheath into the circulation and directly into the right heart. Using fluoroscopy and the steering mechanism the sheath is steered into the coronary sinus vein. As soon as this is achieved the pacing lead and/or guide wire can be advanced out the coronary sinus vein into an appropriate branch in order to achieve left ventricular pacing. This preloaded system and kit streamlines the procedure and thereby facilitates placing pacing leads for biventricular pacing. In addition the kit idea could be used for deploying other pacing leads in other regions.

The description above should not be construed as limiting the scope of the invention to the specific embodiments described, which are provided merely as examples or illustrations. The scope of the invention encompasses interchangeable substitutions that are known to or would be appreciated by those skilled in the art. Many other variations are possible. Thus, the scope of the invention should be determined by the appended claims and their legal equivalents, rather than by only the examples given above.

I claim:

1. A steerable catheter comprising:
a flexible tubular body having a proximal end, a distal end, and at least one lumen;
an inflatable annular balloon positioned on or near the distal end of the tubular body;
at least one electrode positioned on or near the distal end of the tubular body; and
a handle attached to the proximal end of the tubular body,
wherein the handle cooperates with the distal end of the tubular body to steer the catheter.

2. The catheter of claim 1, wherein the tubular body can be rotated and the distal end of the tubular body can be bent to steer the catheter.

3. The catheter of claim 1, wherein the tubular body comprises an inflation lumen in fluid communication with the annular balloon.

4. The catheter of claim 1, wherein there are at least two electrodes.

5. The catheter of claim 1, wherein each electrode has a proximally extending wire that extends through the handle to a connector.

6. The catheter of claim 5, wherein each wire extends through a lumen in the tubular body.

7. The catheter of claim 5, wherein each wire extends through the sidewall of the tubular body.

8. The catheter of claim 1, wherein contrast fluid can be delivered through a lumen in the tubular body.

9. The catheter of claim 1, wherein a guidewire can be placed in a lumen in the tubular body.

10. The catheter of claim 9, wherein the guidewire can be used to facilitate delivery of a pacing lead, defibrillator lead, catheter or stent.

11. The catheter of claim 1, wherein the handle is ergonomic.

12. The catheter of claim 1, wherein the handle has an axial lumen in fluid communication with at least one lumen of the catheter.

13. The catheter of claim 1, wherein at least one steering or pull wire or cord extends proximally from at or near the distal end of the tubular body to the handle.

14. The catheter of claim 13, wherein the handle engages each steering or pull wire or cord and has a mechanism to activate each steering or pull wire or cord to cause the catheter distal end to bend or deflect for steering purposes.

15. The catheter of claim 13, wherein each steering or pull wire or cord extends through a lumen in the tubular body.

16. The catheter of claim 1, wherein the tubular body has a distal section with a radius of curvature and a proximal section and the catheter has means to adjust the radius of curvature of the distal section while maintaining the proximal section substantially straight.

17. The catheter of claim 1, wherein at least one lumen in the tubular body has a diameter of from about 0.01 to about 0.40 inches.

18. The catheter of claim 1, wherein the tubular body has a total length of from about 40 to about 200 cm.

19. The catheter of claim 18, wherein the total length is from about 40 to about 110 cm.

20. The catheter of claim 19 which is intended for access from upper extremity body vessels.

21. The catheter of claim 17, wherein the total length is from about 90 to about 200 cm.

22. The catheter of claim 21 which is intended for access from lower extremity body vessels as well as the upper extremity body vessels.

23. The catheter of claim 1, wherein the distal end of the tubular body can be visualized fluoroscopically to facilitate anatomic placement.

24. The catheter of claim 1, wherein placement of the catheter in the coronary sinus can be confirmed fluoroscopically by the catheter crossing the spine in the left anterior oblique projection and by an atrioventricular electrogram recording confirming this position.

25. The catheter of claim 1, wherein the at least one electrode facilitates recording electrical activity or specific structure potentials.

26. The catheter of claim 1, wherein there are at least two electrodes.

27. The catheter of claim 1, wherein each electrode is annular and has a width of from about 1 to about 4 mm.

28. The catheter of claim 27, wherein two electrodes are positioned about 2 to about 5 mm apart and the inflatable balloon is proximal to these electrodes by from about 1 to about 20 mm.

29. The catheter of claim 27, wherein two electrodes are positioned about 2 to about 5 mm apart, the inflatable balloon is proximal to these electrodes by from about 1 to about 5 mm, and two additional electrodes positioned about 2 to about 5 mm apart are positioned about 2 to about 5 mm proximal to the inflatable balloon.

30. The catheter of claim 1, wherein an array of electrodes is arranged to facilitate mapping, pacing, sensing, and/or catheter ablation of a variety of arrhythmias.

31. The catheter of claim 1 which is available in a variety of different definable curvature capabilities designed to engage the coronary sinus vein or other vessels.

32. The catheter of claim 1, wherein the distal portion of the tubular body can be bent to different radii of curvature.

33. The catheter of claim 31, wherein the catheter may have a secondary bend.

34. The catheter of claim 1, wherein the inflatable balloon is a high pressure balloon.

35. The catheter of claim 34 which is suitable for performing angioplasty.

36. The catheter of claim 33 which is capable of delivering a stent.

37. The catheter of claim 34 which can be used to deliver a stent to a particular stenotic vessel or stenotic corporeal structure, dilating the vessel or stricture, and, optionally, delivering the stent, to resolve an obstruction.

38. The catheter of claim 1, wherein the distal end of the tubular body comprises means for performing catheter ablation.

39. The catheter of claim 36, wherein a plurality of ablation means are aligned along the distal surface of the tubular body in a configuration to create a linear ablation.

40. The catheter of claim 36, wherein a plurality of ablation means are aligned along the distal surface of the tubular body in a configuration to create a circumferential ablation.

41. A catheter for temporary pacing or sensing purposes, which comprises:
a flexible tubular body having a proximal end, a distal end, and at least one lumen;
an inflatable annular balloon positioned on or near the distal end of the tubular body;
at least one electrode positioned on or near the distal end of the tubular body; and
a handle attached to the proximal end of the tubular body,
wherein the handle cooperates with the distal end of the tubular body to steer the catheter and the catheter functions as a temporary pacing or sensing lead.

42. The catheter of claim 41, wherein the catheter functions as a temporary pacing or sensing lead in the coronary sinus vein.

43. A steerable catheter comprising:
a flexible tubular body having a proximal end, a distal end, and at least one lumen and at least one steering or pull wire or cord extending form the distal end to the proximal end; and
a handle attached to the proximal end of the tubular body,
wherein the handle cooperates with the at least one steering or pull wire or cord to steer the distal end of the catheter and at least one lumen facilitates delivery of contrast fluid.

44. The catheter of claim 43, wherein an inflatable annular balloon is positioned at or near the distal end of the tubular body.

45. The catheter of claim 43, wherein at least one electrode is positioned at or near the distal end of the tubular body.

46. The catheter of claim 43, wherein an array of electrodes is arranged to facilitate mapping, pacing, sensing, and/or catheter ablation of a variety of arrthymias.

47. A steerable catheter comprising:
a flexible tubular body having a proximal end, a distal end, and at least one lumen;
at least one radiopaque marker positioned on or near the distal end of the tubular body; and
a handle attached to the proximal end of the tubular body,
wherein the handle cooperates with the distal end of the tubular body to steer the catheter.

48. The catheter of claim 47, wherein the tubular body can be rotated and the distal end of the tubular body can be bent to steer the catheter.

49. The catheter of claim 47, wherein contrast fluid can be delivered through a lumen in the tubular body.

50. The catheter of claim 47, wherein a guidewire can be placed in a lumen in the tubular body.

51. The catheter of claim 50, wherein the guidewire can be used to facilitate delivery of a pacing lead, defibrillator lead, catheter or stent.

52. The catheter of claim 47, wherein the handle is ergonomic.

53. The catheter of claim 47, wherein at least one steering or pull wire or cord extends proximally from at or near the distal end of the tubular body to the handle.

54. The catheter of claim 53, wherein each steering or pull wire or cord extends through a lumen in the tubular body.

55. The catheter of claim 47, wherein the tubular body has a distal section with a radius of curvature and a proximal section and the catheter has means to adjust the radius of curvature of the distal section while maintaining the proximal section substantially straight.

56. The catheter of claim 47, wherein at least one lumen in the tubular body has a diameter of from about 0.01 to about 0.40 inches.

57. The catheter of claim 47, wherein the tubular body has a total length of from about 40 to about 200 cm.

58. The catheter of claim 57, wherein the total length is from about 40 to about 110 cm.

59. The catheter of claim 57, wherein the total length is from about 90 to about 200 cm.

60. The catheter of claim 47, wherein the distal end of the tubular body can be visualized fluoroscopically to facilitate anatomic placement.

61. The catheter of claim 47, wherein placement of the catheter in the coronary sinus can be confirmed fluoroscopically by the catheter crossing the spine in the left anterior oblique projection and by an atrioventricular electrogram recording confirming this position.

62. The catheter of claim 47 which is available in a variety of different definable curvature capabilities designed to engage the coronary sinus vein or other vessels.

63. The catheter of claim 47, wherein the distal portion of the tubular body can be bent to different radii of curvature.

64. The catheter of claim 62, wherein the catheter may have a secondary bend.

65. The catheter of claim 47, wherein the distal end of the tubular body comprises means for performing catheter ablation.

66. The catheter of claim 64, wherein a plurality of ablation means are aligned along the distal surface of the tubular body in a configuration to create a linear ablation.

67. The catheter of claim 64, wherein a plurality of ablation means are aligned along the distal surface of the tubular body in a configuration to create a circumferential ablation.

68. The catheter of claim 47 which can be used for performing a venogram or an angiogram.

69. A steerable introducer sheath comprising:
a flexible tubular body having a proximal end, a distal end, an axial lumen, and at least one axial separation line extending from the proximal end to the distal end;
at least one electrode positioned at or near the distal end of the tubular body;
a hub or handle having a hemostatic seal or valve secured to the proximal end of the flexible tubular body,
wherein the hub has at least one weakened axial line so that it may be manually separated along said at least one axial line.

70. The introducer sheath of claim 69 which has an inflatable balloon at the distal end of the flexible tubular body, said balloon being attached to the flexible tubular body in a manner which permits axial separation of the body.

71. The introducer sheath of claim 69, wherein the hub or handle cooperates with the distal end of the tubular body to steer the sheath.

72. The introducer sheath of claim 69, wherein at least one steering or pull wire or cord extends proximally from at or near the distal end of the tubular body to the handle.

73. The introducer sheath of claim 71, wherein each steering or pull wire or cord extends through a lumen in the tubular body.

74. The introducer sheath of claim 69, wherein the tubular body has a distal section with a radius of curvature and a proximal section and the catheter has means to adjust the radius of curvature of the distal section while maintaining the proximal section substantially straight.

75. The introducer sheath of claim 69, wherein the hub or handle has a primary access port which is axially aligned with the axial lumen of the flexible tubular body.

76. The introducer sheath of claim 69, wherein the flexible tubular body has a length of from about 40 cm to about 200 cm and a lumen diameter of from about 2 F to about 12 F.

77. The introducer sheath of claim 69, wherein a lumen has sufficient diameter to pass a pacemaker or defibrillator lead.

78. The introducer sheath of claim 69, wherein a second axial separation line is formed on the flexible tubular body parallel to said at least one axial separation line.

79. The introducer sheath of claim 69, wherein the hub or handle has two weakened axial lines in alignment with the two axial separation lines on the flexible tubular body.

80. The introducer sheath of claim 78, wherein the two axial separation lines are separated by an arc in the range from about 30° to about 180°.

81. The introducer sheath of claim 80, wherein an inflatable balloon is asymmetrically disposed on the flexible tubular body so that it does not extend into the about 30° to about 180° region between the separation lines.

82. The introducer sheath of claim 80, further comprising an inflation port on the hub and an inflation lumen in the flexible body, wherein the port is connected to the lumen and the lumen is connected to the balloon.

83. The introducer sheath of claim 69, further comprising a flush port on the hub.

84. The introducer sheath of claim 69, wherein the electrodes are shaped so that they remain intact when the tubular body is split.

85. The introducer sheath of claim 83, wherein the electrodes have a half-moon or semicircular shape.

86. A steerable catheter for internal cardioversion or defibrillation, comprising:
a flexible tubular body having a proximal end, a distal end, an axial lumen, and at least one axial separation line extending from the proximal end to the distal end;
an inflatable annular balloon positioned on or near the distal end of the tubular body;
a plurality of electrodes at or near the distal end of the tubular body and proximal to the balloon;
a hub or handle secured to the proximal end of the flexible tubular body,
wherein the distal end of the catheter is advanced into the pulmonary artery or coronary sinus vein to perform internal cardioversion or defibrillation.

87. In an improved introducer sheath comprising an elongated flexible tubular body having a proximal end and a distal end and a hub secured to the proximal end, the improvement which comprises an inflatable balloon mounted on the flexible body near the distal end thereof and means for opening an axial passage along the entire length of the flexible body and hub so that the catheter can be withdrawn over a proximal structure on a device disposed within a body lumen.

88. A kit for implanting a pacing or defibrillator lead comprising:
a catheter of claim 1 and
a pacemaker or defibrillator lead arranged within a lumen of the catheter.

89. The kit of claim 88 which also includes an introducer sheath.

90. The kit of claim 89 which includes two introducer sheaths of different lengths.

91. The kit of claim 88 which includes more than one catheter of claim 1, wherein each catheter has a different pre-formed radius of curvature at its distal end.

92. A kit for implanting a pacing or defibrillator lead comprising:
an introducer sheath of claim 69;
a dilator;
a syringe;
a needle; and
at least one guidewire.

93. The kit of claim 92 which also contains a pacemaker or defibrillator lead.

94. The kit of claim 92 which includes a second introducer sheath.

95. The kit of claim 92 which includes a steerable catheter.

96. The kit of claim 95, wherein the catheter is a steerable catheter of claim 1.

97. A system for implanting a catheter within a patient, comprising:
a first introducer sheath comprising a flexible tubular body having a proximal end, a distal end, and an axial lumen, and a hub having a hemostatic seal or valve secured to the proximal end of the flexible tubular body, and
a second, longer introducer sheath comprising a flexible tubular body having a proximal end, a distal end, and an axial lumen, and a hub having a hemostatic seal or valve secured to the proximal end of the flexible tubular body,
wherein the second introducer sheath can be introduced through the axial lumen of the first introducer sheath and is slidable and rotatable within.

98. The system of claim 97, wherein the flexible tubular body of the first introducer sheath has at least one axial separation line extending from the proximal end to the distal end.

99. The system of claim 97, wherein the hub of the first introducer sheath has at least one weakened axial line so that it may be manually separated along said at least one axial line.

100. The system of claim 97, wherein the flexible tubular body of the second introducer sheath has at least one axial separation line extending from the proximal end to the distal end.

101. The system of claim 97, wherein the flexible tubular body of the second introducer sheath has at least one electrode positioned at or near the distal end of the tubular body.

102. The system of claim 97, wherein the hub of the second introducer sheath has at least one weakened axial line so that it may be manually separated along said at least one axial line,

103. A system for implanting a catheter in a patient, comprising:
a first introducer sheath comprising a flexible tubular body having a proximal end, a distal end, an axial lumen, and at least one axial separation line extending from the proximal end to the distal end; and a hub having a hemostatic seal or valve secured to the proximal end of the flexible tubular body, wherein the hub has at least one weakened axial line so that it may be manually separated along said at least one axial line, and
a second, longer introducer sheath comprising a flexible tubular body having a proximal end, a distal end, an axial lumen, and at least one axial separation line extending from the proximal end to the distal end; at least one electrode positioned at or near the distal end of the tubular body; a hub having a hemostatic seal or valve secured to the proximal end of the flexible tubular body, wherein the hub has at least one weakened axial line so that it may be manually separated along said at least one axial line,
wherein the second introducer sheath can be introduced through the axial lumen of the first introducer sheath and is slidable and rotatable within.

104. A method for implanting a pacemaker lead inside the coronary sinus vein to pace the left ventricle of the heart, which comprises the steps of:
(a) percutaneously inserting an long introducer sheath into the subclavian vein (cepahlic vein or equivalent);
(b) advancing a steerable lumen catheter through the introducer into the right heart;
(c) placing a curve on the catheter once outside of the distal end of the introducer sheath;
(d) moving the catheter backwards and forwards to cannulate the coronary sinus vein under fluoroscopy;
(e) varying the curvature of said catheter while moving it back and forth to find the coronary sinus vein ostium;
(f) optionally injecting puffs of contrast through the lumen of said catheter to help confirm that one is inside the coronary sinus vein;
(g) exchanging said catheter for a different curvature to cannulate the coronary sinus if necessary (i.e., small, medium, large, and extra large curvatures);
(h) advancing a long sheath over the catheter once well inside the coronary sinus vein to have stable access to the coronary sinus;
(i) pulling out the catheter and placing a pacemaker lead into the introducer sheath and directing it into a branch of the coronary sinus vein which is a left ventricular branch;
(j) checking pacing and sensing thresholds of said pacemaker lead and establishing adequate left ventricular pacing;
(k) removing said sheath over the lead while maintaining the lead in stable position within the coronary sinus vein branch; and
(l) securing said catheter to the floor of the pacemaker pocket and attaching the lead to a device to help pace the heart.

105. The method for implanting a pacemaker lead inside the coronary sinus vein as described in claim 100 which includes:
(a) use of a steerable lumen balloon tip catheter;
(b) cannulation and placement of a catheter of claim 1 and advancement of said sheath beyond the coronary sinus ostium to secure the sheath;
(c) pulling back said catheter such that the balloon tip is just outside the introducer sheath which lies in the proximal component of the coronary sinus vein;
(d) inflation of the balloon to occlude the proximal portion of the coronary sinus vein;
(e) injection of contrast through the lumen of said catheter to fluoroscopically image the coronary sinus vein and its branches;
(f) removal of said catheter from the introducer sheath;
(g) placement of a pacemaker lead into said sheath and advancement ventricular branch of the coronary sinus;
(h) testing the pacing and sensing function of said lead to assure thresholds and function;
(i) removal of said sheath over the lead while maintaining the lead appropriate location;
(j) securing the lead end to the floor of the device pocket; and
(k) attaching lead to device.

106. A method for implanting a pacemaker lead inside the coronary n to pace the left ventricle of the heart, which comprises the steps of:
(a) percutaneously accessing an upper chest, extremity or neck vein;
(b) placing an introducer into said vein;
(c) inserting a steerable pacemaker lead catheter system which is d of:
(i) 2-6 F pacemaker lead with an axial lumen and bipolar electrodes (ii) a proximal connector with an internal lumen with locking or stylet or pull cord for steering said lead; and
(iii) a removable handle with a steerable mechanism for pulling and releasing said stylet or pull cord to steer said lead into the branch of the coronary sinus vein;
(d) advancing said steerable lead system with attached handle into said introducer and moving it back and forth with varying curvatures until the coronary sinus ostium is engaged;
(e) optionally administering puffs of contrast to visualize location;
(f) optionally using the electrodes to help define location once connected to an electrogram recording system;
(g) once in the coronary sinus possibly removing the stylet or pull cord and handle and placing a soft steerable guidewire through the central lumen and advancing it into an appropriate coronary sinus branch;
(h) advancing the pacing lead and then following said guidewire to the intended location to pace the left ventricle;
(i) removing the guidewire;
(j) testing the pacing and sensing to confirm appropriateness;
(k) if in step (j) the pacing and sensing are not appropriate, the lead is pulled back and the guidewire is positioned in another location and the procedure repeated until adequate pacing and sensing are achieved;
(l) removing the sheath over the lead while making sure the tip of the lead does not dislodge;
(m) securing the proximal area of the lead to the floor of the device pocket using a lead sleeve or bullet to protect the lead; and
(n) attaching the lead to the device and the remainder of the leads etc.

107. A method for implanting a pacemaker lead inside the coronary sinus vein to pace the left ventricle of the heart as described in claim 106, in which the left ventricular pacing lead uses conventional nonlocking or screw in stylets optionally curved manually to direct the lead into the coronary sinus vein.

108. A steerable catheter comprising:
a flexible tubular body having a proximal end, a distal end, and at least one lumen;
at least one radiopaque marker positioned on or near the distal end of the tubular body; and
a handle attached to the proximal end of the tubular body,
wherein the handle cooperates with the distal end of the tubular body to steer the catheter and the distal end of the catheter forms a bent configuration of greater than at least 0°.

109. The catheter of claim 105, wherein the tubular body can be rotated an the distal end of the tubular body can be bent to steer the catheter.

110. The catheter of claim 108, wherein the distal end of the catheter forms a bent configuration of at least about 180°.

111. The catheter of claim 110, wherein the distal end of the catheter forms a bent configuration of at least about 270°.

112. The catheter of claim 111, wherein the distal end of the catheter forms a bent spiral configuration of at least about 270°.

113. The catheter of claim 108, wherein there are a plurality of lateral openings on the distal end of the tubular body adjacent and/or proximal to the bent configuration.

114. The catheter of claim 108, wherein a guidewire can be placed in a lumen in the tubular body.

115. The catheter of claim 114, wherein the guidewire can be advanced to cause the bent configuration to straighten.

116. The catheter of claim 108, wherein the handle is ergonomic.

117. The catheter of claim 108, wherein at least one steering or pull wire or cord extends proximally from at or near the distal end of the tubular body to the handle.

118. The catheter of claim 117, wherein each steering or pull wire or cord extends through a lumen in the tubular body.

119. The catheter of claim 108, wherein the tubular body has a distal section with a radius of curvature and a proximal section and the catheter has means to adjust the radius of curvature of the distal section while maintaining the proximal section substantially straight.

120. A steerable introducer sheath comprising:
a flexible tubular body having a proximal end, a distal end, an axial lumen, and at least one axial separation line extending from the proximal end to the distal end;
at least one radio-opaque marker positioned at or near the distal end of the tubular body;
a hub or handle having a hemostatic seal or valve secured to the proximal end of the flexible tubular body,
wherein the hub has at least one weakened axial line so that it may be manually separated along said at least one axial line.

121. The introducer sheath of claim 120 which has an inflatable balloon at the distal end of the flexible tubular body, said balloon being attached to the flexible tubular body in a manner which permits axial separation of the body.

122. The introducer sheath of claim 120, wherein the hub or handle cooperates with the distal end of the tubular body to steer the sheath.

123. The introducer sheath of claim 120, wherein at least one steering or pull wire or cord extends proximally from at or near the distal end of the tubular body to the handle.

124. The introducer sheath of claim 122, wherein each steering or pull wire or cord extends through a lumen in the tubular body.

125. The introducer sheath of claim 120, wherein the tubular body has a distal section with a radius of curvature and a proximal section and the catheter has means to adjust the radius of curvature of the distal section while maintaining the proximal section substantially straight.

126. The introducer sheath of claim 120, wherein the hub or handle has a primary access port which is axially aligned with the axial lumen of the flexible tubular body.

127. The introducer sheath of claim 120, wherein the flexible tubular body has a length of from about 40 cm to about 100 cm and a lumen diameter of from about 2 F to about 12 F.

128. The introducer sheath of claim 120, wherein a lumen has sufficient diameter to pass a pacemaker or defibrillator lead.

129. The introducer sheath of claim 120, wherein a second axial separation line is formed on the flexible tubular body parallel to said at least one axial separation line.

130. The introducer sheath of claim 120, wherein the hub or handle has two weakened axial lines in alignment with the two axial separation lines on the flexible tubular body.

131. The introducer sheath of claim 129, wherein the two axial separation lines are separated by an arc in the range from about 30° to about 180°.

132. The introducer sheath of claim 131, wherein an inflatable balloon is asymmetrically disposed on the flexible tubular body so that it does not extend into the about 30° to about 180° region between the separation lines.

133. The introducer sheath of claim 131, further comprising an inflation port on the hub and an inflation lumen in the flexible body, wherein the port is connected to the lumen and the lumen is connected to the balloon.

134. The introducer sheath of claim 120, further comprising a flush port on the hub.

135. The introducer sheath of claim 120, wherein the radio-opaque markers have a half-moon or semicircular shape.
